(19) United States
(12) Patent Application Publication
(10) Pub. No.: US 2005/0028824 A1
(43) Pub. Date: Feb. 10, 2005

(54) Intravaginal fertility plug

(76) Inventors: Lowell Stephen Jordan, Murrieta, CA (US); James Lowell Jordan, Murrieta, CA (US)

Correspondence Address: Lowell Stephen Jordan, 19823 Avenida Rosella, Murrieta, CA, 92562, US

(21) Appl. No.: 10/939,446

(22) Filed: Sep. 14, 2004

Related U.S. Application Data
(63) Continuation of application No. 10/291,255, filed on Nov. 12, 2002, now abandoned.

(51) Int. Cl. A61K 9/14; A61F 6/06

(52) U.S. Cl. 128/830

(57) ABSTRACT

This invention relates to an intravaginal fertility plug, for use by both humans and animals, constructed from regions consisting of impermeable and permeable, polymeric film membrane covering, completely or partially, an elastically deformable and compressible interior, which, because of pressure from the vaginal walls on the unit (forming a seal), can be used to promote fertility by temporarily sealing in semen in the female reproductive tract. A lubricant can be used with the intravaginal fertility plug to facilitate insertion into and removal from the vagina; the lubricant would enhance the seal formed between the unit's surface and the vaginal wall and may contain medication itself. The intravaginal fertility plug, not intended for overly prolonged use in the vaginal should be one-time use, and can, upon removal, be discarded.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### BACKGROUND OF THE INVENTION

1. Filed of Invention

This invention relates to an intravaginal fertility plug, lubricated or nonlubricated, for use by both humans and animals, constructed from an impermeable polymeric film membrane, partially covering an elastically deformable and compressible interior, which, because of pressure from the vaginal walls on the unit (forming a seal), can be used to prevent escape of semen from the vagina immediately after ejaculation or injection of semen into the female's reproductive tract, but instead remain in the vicinity of the cervix (or in the uterus) available for fertilization. The intravaginal fertility plug, not intended for overly prolonged use in the vagina, can, upon removal, be discarded.

2. Background of the Invention

Infertility —The Problem and Interventions used. Approximately ten percent of American couples are infertile; of those who desire to be fertile, 85 to 90% are treated with conventional medical therapies such as medication or surgery. Both surgery and medication therapies can pose health risks. For example, the National Cancer Institute has reported that fertility drugs may increase the risk of ovarian cancer. Fertility drugs can also be expensive and beyond the financial means of many people, particularly if those medications are not covered by medical insurance or HMO's. Other medical procedures to correct infertility can also be expensive. The average cost of an IVF cycle in the, U.S. is 12,400. Moreover, the success rate for IVF is about 29.1 percent.

Males and females are equally responsible for the incidence of infertility. For males, that may bean a concentration of sperm in the semen of less than 20 million per milliliter or less than 50% motility at one hour (after ejaculation). The problem with male infertility is that much of the semen is lost after being placed into the vagina. Thus, if more sperm were to remain in the vagina, the number of sperm available to cause impregnation would increase. This would be accomplished through mechanical means, such as a plug, and not by either surgical or medical interventions. The plug would have to be intravaginal and conform to the vaginal walls to form a seal against semen loss from the vagina.

Intravaginal Devices —Introduction. Intravaginal devices are not new. For years, they have been used to absorb menstrual blood or as suppositories to deliver medicines. Intravaginal devices to introduce medications include a plug (Zaffaironi, U.S. Pat. No. 3,854,480), an inflatable device (Donnell and Brown, U.S. Pat. 5,299,581), a device with a tube for introducing medicines (Murray, U.S. Pat. 1,355,846), and a device with a soluble shell (Martocci-Pisculi, U.S. Pat. No. 1,575,123).

Zaffaraoni (U.S. Pat. No. 3,854,480) and Donnell and Brown (U.S. Pat. No. 5,299,581) both developed intravaginal devices utilizing a plug with a permeable polymeric membrane and elastically deformable interiors to deliver medications, intravaginally, to a female and to absorb bodily fluids. Zaffaroni (U.S. Pat. No. 3,854,480) developed a drug-delivery system having the following claims. First, the patent contains “a solid inner matrix having solid particles of said drug dispersed therethrough, and inner matrix being permeable to passage of said drug by diffusion.” Second, it contains the claim of “an outer polymeric membrane, insoluble in body fluids, surrounding said inner matrix, said polymeric membrane characterized by being formed of a drug release rate controlling material which is permeable to passage of said drug by diffusion but at a rate which is lower than through said inner matrix material.” The polymeric membrane claimed “is an ethylene-vinyl acetate polymer” and the “inner matrix material is silicone rubber.” That the membrane is permeable is noted in claims 1(b), 1(c), 3(b), 3(c), 10(b), 10(c), 11(b), and 11(c). The membrane is permeable and the “inner mass transfer of . . . said drug [progesterone] dispersed therethrough, said inner material being permeable to passage of said drug by diffusion” and, thus, would not serve to enhance fertility by stopping the loss of semen from the vaginal tract. An intravaginal fertility plug would have to have an impermeable polymeric membrane at the end which is inserted into the vagina. Thus, a membrane system, such as that used by Zaffaraoni (U.S. Pat. No. 3,854,480) being permeable (necessary to deliver medications) would not block loss of semen from the vagina. One cannot infer from Zaffarani's (U.S. Pat. No. 3,854,480) device, designed to deliver a substance into the vagina as having the same nature as one designed to block loss of a substance from the vagina.

Donneil and Brown (U.S. Pat. No. 5,299,581) developed a drug-delivery system “which includes a shield of relatively thin, flexible, membrane-like material such as a plastic that surrounds a pressure element, receives medicaments, holds the medicaments against the vagina walls and prevents their inadvertent contact with the pressure element or tampon material prematurely.” Donnell and Brown (U.S. Pat. No. 5,299,581) also indicate that the medicament may “be placed as a glob on the compacted device. The device may be inserted into the vagina by the means of the rod, the rod withdrawn and the device inflated.” Moreover, “in the preferred construction, the filler material for the bulb is gaseous. . . . Such a gas would be air or carbon dioxide.” Thus, Donnell and Brown (U.S. Pat. No. 5,299,581) relies upon inflation. This carries with it notable risks to the user. Air under pressure, when introduced into the vagina, can be fatal. Cases have existed that air was blown into the vagina, during cunninglitis, and a fatal air embolism result afterward. The current invention does not use inflation to accomplish the desired objectives of introducing medications into the vaginal tract. The problem would exist with a closed device, impermeable to air, that would be introduced into the vagina. A release mechanism is required so that air is not forced against the vaginal walls.

Moreover, the permeable nature throughout would be contraindicative to its used as a plug, such as stopping the loss of semen and moisture from the vaginal tract. Neither a permeable system as developed by Zaffaroni (U.S. Pat. No. 3,854,480) or an inflatable system as developed by Donnell and Brown (U.S. Pat. No. 5,299,581) can both deliver a substance into the vagina while blocking the loss of substance from the vagina. The functions are inherently contraindicated. They also cannot serve as a plug that not only restricts loss of semen, but also does not introduce a pressurized system, and thereby risks, into the female. While a unit with a completely impermeable polymeric membrane is possible, and appear to be similar to the plug developed by Zaffarini (U.S. Pat. No. 3,854,480), the device would end up simply being a balloon, filled with a matrix (or gas). It would not truly conform comfortably to the female vagina. To conform to the vagina, pressure from within the intravaginal fertility plug would have to released outside of the female. That can be accomplished by not having a seal at the end of the plug not inserted into the female or by having a different, permeable polymeric membrane at that end. Thus, an intravagainal fertility plug must be able to block sperm, and it's accompanying moisture, from escaping (necessitating one end having a impermeable polymeric membrane), conform to the vagina (necessitating the contents to be deformable and elastic), and capable of release of internal pressure outside of the female (necessitating the other end to be either open, without any membrane covering, or permeable to gas and/or excess fluid loss). These prerequisites for a successful intravaginal fertility plug were not met in prior inventions, but have been successfully met in this invention, the intravaginal fertility plug.

Intravaginal device to block semen loss. An intravaginal device would be useful to prevent semen loss from a female after it has been deposited into her vagina. Immediately after ejaculation, approximately 90% of the ejaculate is expelled from the female, through her vagina, and thus is unavailable for fertilization (since sperm outside of the female serve no reproductive function). Thus, even though a male may ejaculate the hundreds of millions of sperm needed to be considered fertile, the loss of most of the sperm would put his sperm left in the female at what may be infertile levels. If a male is only marginally fertile, the loss of such sperm could result in the same fertility ability as if he was actually infertile. For example, if 300 million sperm are deposited, 270 milion may be lost by expulsion from the vagina. But, if the human male produced only 30 million sperm in his ejaculate, he would be considered to be infertile. Thus, if a plug could retain the 30 million sperm of the otherwise infertile male, he would have as many sperm available for fertilization as a male who deposits sperm at a rate of 300 million sperm, but with 270 million of them expelled from the female's reproductive tract. For the sperm to be retained in the vagina, an invention with an impermeable polymeric membrane would have to be available for use to promote conception. The impermeable nature is essential since absorption of fluids would dessicate the semen.

Moreover, even the sperm in the vicinity of the cervix encounters a barrier, both physical and chemical. It takes a sufficient number of sperm to penetrate the cervical mucous. By enhancing the number of sperm crossing the barrier, fertility may be enhanced. From a chemical standpoint, ejaculate is slightly basic, but the vaginal secretions are slightly acidic (which is toxic to sperm). Because the sperm's environment shifts from a basic pH to an acidic one after about an hour after ejaculation, for fertilization to occur, the sperm must enter the uterus within an hour after being deposited in the female. If the semen is not so dissipated, as occurs when it is expelled from the vagina, the pooling of the semen would help prolong the time during which the sperm exist in a ph-friendly environment. All these factors indicate the need for the development of an intravaginal fertility plug which would be placed into the vaginal barrel immediately after semen has been placed therein, either through coitus or through artificial isemnation, and left therein for an hour after semen introduction into the female reproductive tract, so that the sperm may remain where they are available for fertilization and not lost by being expelled from the vagina. An intravaginal fertility plug would have increased fertility enhancement qualities, if it was to be coated with a lubricant that would help seal the plug against the vaginal walls, while providing a pH buffering, moisture, nutrient, and electrolyte balance favorable to semen viability and motility. Such a lubricant could also be applied prior to the semen being deposited into the female's reproductive tract.

To date, medical research and inventions covering semen in a vaginal tract have been directed at keeping the sperm out of the vagina (e.g., condoms) or away from the cervix (e.g., diaphragms and cervical caps). However, for the 30% of human couples who are involuntarily infertile, the problem is not one of contraception, but of conception. In those couples, about 40% are infertile because the male has too low of a sperm count and about 40% are from female factors (such as a cervix which does not permit for the passage of sperm into the uterus). For both low sperm count and cervical causes of infertility, the use of an intravaginal plug to keep the sperm that are deposited would raise the sperm count available for fertilization and able to cross the cervical barrier and mucous. The use of an intravaginal plug is also adaptable to the insertion of medications before or after semen introduction into the female reproductive tract. The medications and or spermiphillic substances can be incorporated into an intravaginal fertility plug lubricant; but they would not be within the plug itself

Even with artificial insemination, leakage of the injected fluid, containing the sperm, occurs almost immediately after deposition has taken place. Thus, the number of sperm available for fertilization is quickly decreased because of expulsion from the female. Even then an intravaginal fertility plug would be useful. Not only would such an intravaginal fertility plug prove useful for humans desiring offspring, but can also be used in the breeding of animals (in which a semen sample can be quite costly). Because of the need for a plug to keep semen inside a female, to enhance the possibility of fertilization, this invention, an intravaginal fertility plug, has been developed. No invention has been developed, patented, or described in the literature, which serves as an intravaginal intravaginal fertility plug. Whereas an intrauterine device (described later) has been developed, no intravaginal device has been developed to retain semen in the female reproductive tract.

In terms of the design of the plug itself the inventions by Zaffaraoni (U.S. Pat. No. 3,854,480) and Donnell and Brown (U.S. Pat. No. 5,299,581) come closest. The first is an intravaginal plug with a permeable polymeric membrane and elastically deformable interiors to deliver medications, intravaginally, to a female. The second uses a tampon, impermeable polymeric membrane shield, and inflation (with a gas). Donnell and Browns cannot serve to both block the loss of sperm while simultaneously delivering medications, lubricants, or materials to facilitate sperm survival. They also cannot block loss of semen while remaining permeable to facilitate compression by the vaginal walls.

The current invention utilizes a plug with polymeric membrane which surrounds the elastically deformable interior. Thus, the current invention can be designed to deliver medication or absorb bodily fluids from targeted locations on the vagina, the cervix, or both, while being capable of blocking sperm loss. Moreover, it can be modified to permit addition of sperm-friendly fluids, besides those already produced by the male and female, possibly helping the sperm survive. Because the sperm would be in direct contact with an impermeable part of the intravaginal device, the current invention's impermeable polymeric membrane is necessary not only to prevent passage of sperm from the vagina, but also to prevent dehydration of semen which would result from the absorptionof fluids into the plug. The internal component, being enclosed within an impermeable polymeric membrane, must be sterile; but, if desired, delivery of medications becomes possible through the permeable portion. Medications can also be delivered with this invention externally to the unit.

Because the current invention can transmit medications intravaginally to targeted locations in the female reproductive tract (contained within an internal matix) and also has an impermeable polymeric membrane component, the prior art (in U.S. Pat. No. 3,854,581 and 5,299,581) would be rendered unsatisfactory for its intended purpose (the application of medications intravaginally), then there is no suggestion or motivation to make the proposed modification, as existent in this invention, and therefore it is not an obvious modification (In re Gordon, 733 F. 2d 900, (Fed. Cir. 1984). As such, the current invention is not only novel in terms of utility, but also in structure and design.

Donnell (U.S. Pat. No. 3,858,571) sought to address retention of sperm in the female reproductive tract by the use of a coorual plug; his invention was intended to “obstruct entry to the Fallopian tubes of an overall bullet configuration having a cone-shaped forward portion that coopreates with the uterine walls and a wire anchoring means extending from about the mid-portion of the plug to secure the plug in position. The cornual plug is optionally provided with a pendant string or cord for removal of the device, thus, the device is useful for sterilization or contraception.” Because it is an intrauterine device, the invention by Donnell requires the services of a physician to put into place. Moreover, being intrauterine, difficulties may arise from its removal or from its string serving as a means for microorganisms to travel into the uterus and fallopian tubes. The difficulties of his invention also make it more costly to use and not usable by non-physicians (e.g., the females themselves) or non-veterinarians (e.g., farmers, animal breeders). An intrauterine device, particularly if equipped with a string to remove it, serves as a conduit for microbes to enter the uterus and possibly directly into the fallopian tubes. In contrast, the invention described herein can be utilized by the woman herself (or her mate) or be put into place into animals by farmers and non-veterinarians. By being intravaginal, the current intravagainal device would present less trauma to the female reproductive system, be less time dependent after introduction of sperm into the female's reproductive tract, and be less painfll, than the more intrusive approach of placing a device to block fallopian tubes. Finally, by being intravaginal, rather than intrauterine, insertion can be done irmmediately after coitus, or artificial introduction of semen into the female, without the assistance of licensed, trained professionals; thus, an intravaginal device would be more private to use and facilitate intimacy when used between two persons desiring fertilization.

An intravagainal fertility plug must be able to block sperm from escaping (necessitating one end having a impermeable polymeric membrane), conform to the vagina (necessitating the contents to be deformable and elastic), and capable of release of internal pressure outside of the female (necessitating the other end to be either open, without any membrane covering, or permeable to gas and/or fluid loss). These prerequisites for a successful intravaginal fertility plug were not met in prior inventions, but have been successfully met in this invention, the intravaginal fertility plug. Thus, the current invention is novel (from the patents of Zaffaraoni (U.S. Pat. No. 3,854,480) and Donnell and Brown (U.S. Pat. No. 5,299,581)) in that it utilizes (1) an impermeable polymeric membrane and a permeable polymeric membrane/ or lack of any membrane (in different regions of the device), and (2) the capacity to permit reduction in unwanted, hazardous, uncomfortable, and/or unnecessary intravaginal pressure by permitting its escape outside of the female. Moreover, it may incorporate a string with it without having to be placed intrauterine. Thus, the current invention, being intravaginal, is less traumatic to the female, easier to apply, use, and remove than the intrauterine device by Donnell (U.S. Pat. No. 3,858,571); it also has no conduit possibilities of microbes from the vagina directly to the fallopian tubes. Because it is more functional in use, it becomes cheaper to use and may no longer be dependent upon the services of a physician or veterinrian to insert or remove.

The current invention also incorporates the use of incomplete coverage of the elastically deformable and compressible interior. By doing so, the non-covered end permits greater ease of compression and expansion of the device, since a membrane (in U.S. Pat. No. 3,854,581 and 5,299,581) would itself restrict optimal compression, elasticity, and compression. A prior patent application by James Jordan (20040089312) was abandoned simply because the use of a completely sealed fertility aid would not satisfactorily conform to the vaginal walls. The devise would act as a balloon, the lack of pressure release was not only contrary to the molding of the plug to the vaginal walls; it also increased the level of discomfort. An intravaginal fertility plug would have to incorporate the vaginal dynamics, the semen pool and comfort of the user. Unlike previous fertility or intravaginal devices, this invention does take those factors into consideration. The open end along with the elastically deformable and compressible interior allow conformance to the vagina, while the impermeable polymeric membrane prevents loss of semen and excess moisture. Thus, the current intravaginal fertility plug has greater flexibility in use than would be present if it used a membrane, permeable or impermeable, which covered the entire device. It satisfies the major components of success for such an invention.

Namely, an intravagainal fertility plug must be able to block sperm from escaping (necessitating one end having a impermeable polymeric membrane), conform to the vagina (necessitating the contents to be deformable and elastic), and capable of release of internal pressure outside of the female (necessitating the other end to be either open, without any membrane covering, or permeable to gas and/or fluid loss). These prerequisites for a successful intravaginal fertility plug were not met in prior inventions, but have been successfullly met in this invention, the intravaginal fertility plug.

Prior Art References Related to Invention (as per IDS/PTO-1449)

| Document Number | Date | Name | Class | Subclass |
|---|---|---|---|---|
| 3,854,480 | 12-1974 | Zafforoni | 128 | 260 |
| 3,858,571 | 1-1975 | Rudolph | 128 | 831 |
| 5,299,581 | 4-1994 | Donnell and Brown | 128 | 830 |
| 20040089312 | 5-2004 | Jordan | | |

U.S. Pat. Docs. Cited in Background of Invention

| | | | | |
|---|---|---|---|---|
| 3,858,571 | 1-1975 | Rudolph | 128 | 831 |
| 5,299,581 | 4-1994 | Donnell and Brown | 128 | 830 |

### Background of Invention—Objects and Advantages

Accordingly, several objects and advantages of the present Patent Application of Lowell S.Jordan and James Lowell Jordan for the “Intravaginal fertility plug” invention are:

(a) to permit the enhancement of fertility, in a number of cases, without having to rely upon hormone injections or other medical interventions;

(b) to enhance the number of sperm remaining in a female's reproductive tract after coitus or artificial insemination, increasing the likelihood of successful fertilization;

(c) to provide a mechanical means to increase the fertility of many couples without having to rely upon hormonal treatments or other invasive procedures;

(d) to assist couples desiring children to have greater control over their childbearing futures;

(e) to assist animal breeders, veterinarians, farmers and others who rely upon artificial insemination of animals to decrease the costs of purchasing semen which does not resul in fertilization;

(f) to provide an aid to increased fertilization that is easy to use, inexpensive to manufacture and purchase, and is disposable;

(g) to provide a means for Roman Catholics and persons of other religious faiths, who are prohibited from engaging in some means of enhancing their fertility (e.g., artificial insemination) without violating their religious beliefs;

(h) to reduce the choice of having to rely upon an external sperm donor, for infertile couples, when such may constitute adultery according to their religious fith;

(i) to permit, as the situation may arise, for the use of lower amounts of semen for artificial insemination, particularly for animals, and thus allowing for more economical use of purchased semen;

(j) to enhance the intimacy of coitus performed (when done by humans) with the intent to conceive by enhancing the privacy and cooperation of the two persons;

(k) the intravaginal fertility plug can be made sterile and its sterility maintained by being put into separate packages;

(l) the intravaginal fertility plug can be inserted into the vagina by the use of already developed and marketed tampon applicator;.

(m) the intravaginal fertility plug, and the applicator (if used), can be coated with a nonspermicidal lubricant facilitating insertion into and removal from the vagina;

(n) a nonspermicidal lubricant would help form a seal between the outside membrane of the unit with the vaginal wall so that loss of semen is further minimzed;

(o) a nonspermicidal lubricant can be included with the internal matrix to faiciiate insertion into and removal from the vagina;

(p) overall size and dimensions of invention would correspond to the actual application in terms of the individual female, use with medications, targeted area in the reproductive tract to be medicated, after coitus or artificial insemination, or species involved;

(q) to facilitate its sealing function, without interfering with the cervix, the plug may be shorter than the relaxed vagina;

(r) the device can be equipped with a string or have the membrane longer than the elastically deformable and compressible interior.

### BRIEF SUMMARY OF THE INVENTION

In accordance with the present invention an intravaginal fertility plug comprising of impermeable and permeable polymeric membranes, in different regions of the invention, enclosing a compressible—elastically deformable—interior, so that immediately after deposition of semen into the female, the unit can be inserted into the vagina (by a tampon-like applicator if desired); thereupon, the female's vaginal muscles compress the unit forming a plug which restricts or prohibits the expulsion of the semen from the vagina and helps keep the sperm in the vicinity of the cervix for entry into the uterus for fertilization—without dehydrating it — or adversely affecting semen viability or motility. A nonspermicidal lubricant can be used, external to the membrane or within the internal matrix, to ease insertion into and removal from the vagina; the lubricant also serves to help seal the intravaginal fertility plug in the vagina, thus helping prevent the loss of semen. The lubricant can also be of a pH, buffering capacity, moisturizing, nutrient or electrolyte composition to help promote sperm viability and motility.

### DRAWINGS—FIGURES

The above, and other embodiments of the present invention, may be more fully understood from the detailed description taken together with the accompanying drawings illustrating examples wherein sinnar reference characters refer to similar elements in which:

FIG. 1 illustrates an intravaginal fertility plug from a sideways, sectional view showing an impermeable polymeric membrane longer than the enclosed elastically deformable and compressible interior, but not covering one end of the elastically deformable and compressible interior;

FIG. 2 illustrates an intravaginal fertility plug from a sideways, sectional view showing an impermeable polymeric membrane the same length as the enclosed elastically deformable and compressible interior, but not covering one end of the elastically deformable and compressible interior;

FIG. 3 illustrates an intravaginal fertility plug from a sideways, sectional view showing an impermeable polymeric membrane the same length as the enclosed elastically deformable and compressible interior, with the one end of the elastically deformable and compressible interior covered with a permeable polymeric membrane;

FIG. 4 illustrates an intravaginal fertility plug from a cross-sectional view showing an impermeable polymeric membrane surrounding the elastically deformable and compressible interior.

### DRAWINGS—Reference Numerals

1 Elastically deformable and compressible interior

2 Permeable polymeric membrane

3 Impermeable polymeric membrane

### DESCRIPTION OF THE PREFERRED EMBODIMENTS

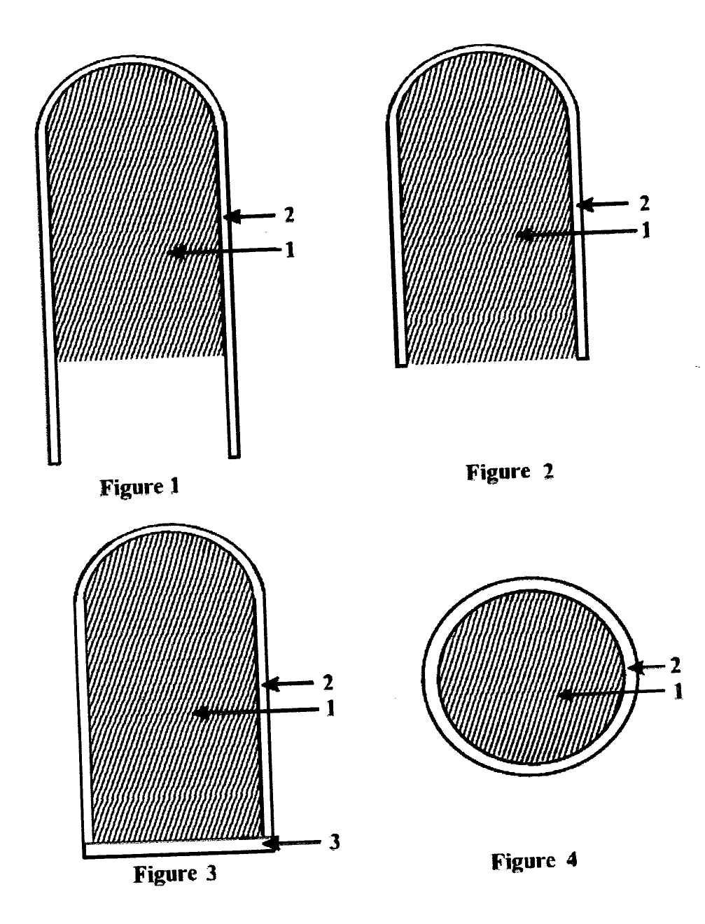

Referring now to the Drawings, there is shown in FIG. 1 illustrates an intravaginal fertility plug from a sideways, sectional view showing an (2) impermeable polymeric membrane longer than the enclosed (1) elastically deformable and compressible interior, but not covering one end of the (1) elastically deformable and compressible interior. FIG. 2 illustrates an intravaginal fertility plug from a sideways, sectional view showing an (2) impermeable polymeric membrane the same length as the enclosed (2) elastically deformable and compressible interior, but not covering one end of the (2) elastically deformable and compressible interior. FIG. 3 illustrates an intravaginal fertility plug from a sideways, sectional view showing an (2) impermeable polymeric membrane the same length as the (1) enclosed elastically deformable and compressible interior, with the one end of the (1) elastically deformable and compressible interior covered with a (3) permeable polymeric membrane. FIG. 4 illustrates an intravaginal fertility plug from a cross-sectional view showing an (2) impermeable polymeric membrane surrounding the (1) elastically deformable and interior.

The (2) impermeable polymeric membrane and/or the (3) permeable polymeric membrane is a polymeric membrane, being made of from a number of materials including latex rubber and mixtures thereof cellulose acetate plastic, vinyl plastic, polyethylene plastic, polypropylene plastic, polyvinyl chloride plastic, polyvinyl acetate plastic, polystyrene plastic, polymethyl methyl-acrylate plastic, polyacrylonitrile plastic, vinyllite plastic, saran plastic, polytetraflouroethylene plastic, poiytriflourochloroethylene plastic, polycaprolactam plastic, polyester plastic, urea formaldehyde plastic, polyurethane plastic, isotactic polypylene plastic, nylon plastic, rayon plastic, polyamide plastic, phenolic plastic, silicone plastic, silk fiber, cotton fiber, cellulose fiber, wool fiber, animal skin, animal intestinal tissue, animal conmective tissue, metallic fiber, mineral fiber, any manmade material, any plant material, any animal material, and miutures thereof. Moreover, when used to enhance fertility, the (2) impermeable polymeric membrane and/or permeable polymeric membrane can be coated with a nonspermicidal lubricant to facilitate entry into and removal from the vagina. They can also be coated with a gel, foam, or lubricant containing medication(s). If the device is used to keep semen in the vagina, impermeability—the (2) impermeable polymeric membrane—of a portion of the intravaginal fertility plug is important since a permeable polymeric membrane would serve to dessicate the semen.

The (1) elastically deformable and compressible interior would consist of any elastically compressible material, including (but not limited to) cellulose fiber (e.g., cotton), cotton fiber, any natural or synthetic or sponge (made from any natural or synthetic material), any natural or synthetic compressible solid foam, or other material capable of being elastically deformed. When used to block loss of semen from the vagina, the (1) elastically deformable and compressible interior serves to push the impermeable polymeric membrane against the walls of the vagina, helping form a seal in the vagina. Actual length of the intravaginal fertility plug would be less than the length of the relaxed vagina so that the plug would not be pressed against the cervix, causing it to act as a barrier against fertilization. For humans, the average relaxed vagina may have a length of 2 to 3 inches, but cases of 6 inches or more have been recorded. Thus, length would have to conform to the female involved, the length required for a particular female most likely requiring a determination by a physician or other health professional. Diameter of the plug also depends upon whether the use is for post-coital use or for artificial insemination. For humans, the diameter after coitus may approximate that of an erect penis since the vagina has already conformed to that size. But, it may be smaller, especially when used in conjunction with artificial insemination. For use with animals, the intravaginal fertility plug would have to

be in accordance with the species and specific breed involved. Size (diameter and length) are also considerations for the intravaginal fertility plug that are influenced by species and/or breed when used to deliver medications intravaginally.

1: An intravaginal fertility plug consisting of
claim 1(a): external region;
claim 1(b): elastically deformable and compressible interior.

2: The external region may possess:
claim 2(a) impermeable polymeric membrane;
claim 2(b) permeable polymeric membrane;
claim 2(c) no membrane;

3: Any of the following may be used as the impermeable polymeric membrane in this invention, as per claim 1: latex rubber and mixtures thereof, cellulose acetate plastic, vinyl plastic, polyethylene plastic, polypropylene plastic, polyvinyl chloride plastic, polyvinyl acetate plastic, polystyrene plastic, polymethyl methyl-acrylate plastic, polyacrylonitrile plastic, vinyllite plastic, saran plastic, polytetraflouroethylene plastic, polytriflourochloroethylene plastic, polycaprolactam plastic, polyester plastic, urea formaldehyde plastic, polyurethane plastic, isotactic polypylene plastic, nylon plastic, rayon plastic, polyamide plastic, phenolic plastic, silicone plastic, silk fiber, cotton fiber, cellulose fiber, wool fiber, animal skin, animal intestinal tissue, animal connective tissue, metallic fiber, mineral fiber, any marnade material, any plant material, any animal material, and mixtures thereof

4: Any of the following may be used as the permeable polymeric membrane in this invention, as per claim 1: latex rubber and mixtures thereof, cellulose acetate plastic, vinyl plastic, polyethylene plastic, polypropylene plastic, polyvinyl chloride plastic, polyvinyl acetate plastic, polystyrene plastic, polymethyl methyl-acrylate plastic, polyacrylonitrile plastic, vinyllite plastic, saran plastic, polytetraflouroethylene plastic, polytriflourochloroethylene plastic, polycaprolactam plastic, polyester plastic, urea formaldehyde plastic, polyurethane plastic, isotactic polypylene plastic, nylon plastic, rayon plastic, polyarnide plastic, phenolic plastic, silicone plastic, silk fiber, cotton fiber, cellulose fiber, wool fiber, animal skin, animal intestinal tissue, animal connective tissue, metallic fiber, mineral fiber, any mamnade material any plant material any animal material, and ixures thereof.

5: Any of the following may be used as the elastically deformable and compressible interior of this invention, as per claim 1: cotton fiber, any natural or synthetic sponge, any natural or synthetic compressible solid foam, or other material capable of being elastically deformed.
